(19) United States
(12) Patent Application Publication
(10) Pub. No.: US 2016/0184733 A1
(43) Pub. Date: Jun. 30, 2016

(54) METHOD FOR SEPARATION OF A MOLECULAR SPECIES BY SUBLIMATION

(71) Applicant: Dow Global Technologies LLC, Midland, MI (US)

(72) Inventors: Edward L. Cussler, Edina, MN (US); Matthew L. Grandbois, Midland, MI (US); Russell J. Holmes, Chanhassen, MN (US); Nathan T. Morgan, St. Paul, MN (US); Yi Zhang, Witchita, KS (US)

(21) Appl. No.: 14/958,991

(22) Filed: Dec. 4, 2015

Related U.S. Application Data
(60) Provisional application No. 62/093,045, filed on Dec. 17, 2014.

(51) Int. Cl. B01D 7/00; C07D 403/14; C07C 209/86

(52) U.S. Cl. CPC B01D 7/00; C07C 209/86; C07D 403/14

(57) ABSTRACT

The present invention provides a method for separation of a targeted molecular species with high purity from a mixture, by controlling temperatures of sublimation unit calculated by a specific formula.

### FIELD OF THE INVENTION

The present invention relates to a method for separation of a molecular species by sublimation. More particularly, the present invention relates to a method for separation of a targeted molecular species with high purity from a mixture of molecules by controlling temperatures of sublimation unit calculated by a specific formula.

### BACKGROUND OF THE INVENTION

Methods for separation of a molecular species from a mixture are known to include without limitation distillation, column chromatography, recrystallization and sublimation. Sublimation is used in the electronics industry for the purification of small molecules including those used in organic light emitting diodes (OLED).

JP5525677B discloses a sublimation apparatus comprising a purification unit, a thermal control unit located on and generating a thermal gradient to the purification unit, a gas exhaust unit and a gas supply unit, in which a carrier gas is heated before it is supplied to the purification unit. It also discloses that the thermal gradient is gentle and continuous because of the heated carrier gas, thus impurities and the target substance deposit with sufficient distance from each other. JP4866527B discloses a method for purification with a sublimation apparatus, wherein a purification zone of the apparatus is heated by electromagnetic induction. The thermal gradient of the apparatus is controlled like steps, i.e. the thermal gradient is flat during each material deposits. However, those prior arts do not disclose how to determine the thermal gradient which is sufficient for the separation of the target substance from impurities.

Small organic molecules used in the electronics industry are required to have high purity. However, quantitative determination of the thermal gradient remains more art than science. As a result, sublimation units of apparatuses are often wastefully long, or the purity of the obtained molecules is not satisfactory. Therefore, a method to effectively separate molecules, such as OLEDs, from a mixture with isolated high purity is still desired.

### SUMMARY OF THE INVENTION

Inventors of this invention have found a method to effectively separate a molecular species from a mixture wherein the separated material is of high purity. One aspect of this invention relates to a method for the separation of targeted molecular species from a mixture of two or more species by a sublimation system comprising (A) a thermal sublimation unit with a linear tube, comprising (A-1) a sublimation part where the mixture is heated and sublimated and (A-2) a deposition part where sublimated molecules are deposited, in which the targeted molecular species is deposited in a specific area between position X and position Y in the deposition part and (B) thermal control units which control the temperatures of multiple positions of the thermal sublimation unit to decrease as the distance from the sublimate part increases, wherein position X, position Y and temperatures of the thermal sublimation unit are controlled to satisfy the following conditions (i) to (iii):

(i) the temperature of the sublimation part is at least higher than the sublimation temperature of the targeted molecule species,
(ii) position X is away from deposition areas for molecules with higher deposition temperatures than the targeted molecular species, and the temperature at the position X is set at the deposition temperature of the targeted molecular species,
(iii) position Y is away from the deposition area of the targeted molecular species, and the temperature at position Y is higher than deposition temperatures of molecular species with lower deposition temperatures than the targeted molecular species.

In this method, the deposition areas are calculated by deposition distances for each of the molecular species using formula (1)

N₍ₓ₎=N₍₀₎e^{-αx} (1)

wherein x is a distance from beginning of deposition for a molecular species to be calculated, N(0) is the amount of material deposited at x=0, N(x) is the amount of material deposited at the distance x and α is the sticking coefficient of the molecular species.

Another aspect of this invention relates to a method for separation of two or more molecular species from a mixture of the molecular species by sublimation, wherein the method comprises the steps of: (a) calculating deposition temperatures for each of the molecular species at a particular pressure from vapor pressure curves of each molecular species, and determining the order between the molecular species in the order of decreasing deposition temperatures, (b) calculating the sticking coefficient of a molecular species which has the highest deposition temperature within a mixture to be separated, (c) calculating a deposition distance for the molecular species with the highest deposition temperature from formula (1)

N₍ₓ₎=N₍₀₎e^{-αx} (1)

wherein x is a distance from material deposited at x=0, N(x) is the amount of material deposited at the distance x, and α is the sticking coefficient of the molecular species, (d) determine a deposition point for the molecular species which has the next highest deposition temperature, wherein the deposition point is away from at least the deposition distance of the molecular species with the highest deposition temperature, (e) repeating steps (b) to (d) for the next molecular species which has the next highest deposition temperature until deposition points for all molecular species are determined, (f) setting temperatures of each deposition point based on the deposition temperatures of the corresponding molecular species, and (g) conducting separation by sublimation.

### BRIEF DESCRIPTION OF THE DRAWINGS

FIG. 1 is a sectional view showing a thermal sublimation system 1 of one aspect of this invention.

FIG. 2A is a figure which shows the deposition curves of NPD and TCTA versus distance and temperature of deposition unit, obtained in Example 1.

FIG. 2B is a figure which shows the relationship between distance and product composition obtained in Example 1.

FIG. 3A is a figure which shows the deposition curves of NPD and TCTA versus distance and temperature of deposition unit, obtained in Example 2.

FIG. 3B is a figure which shows the relationship between distance and product composition obtained in Example 2.

### DETAILED DESCRIPTION OF THE INVENTION

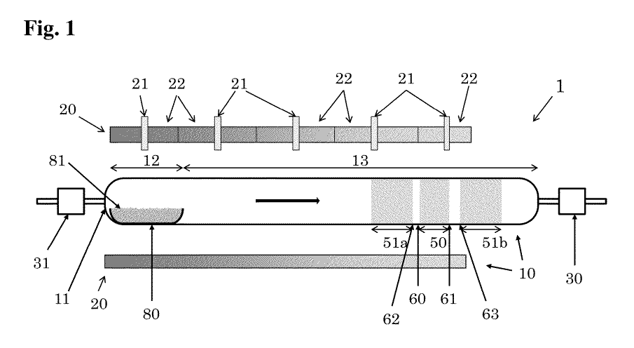

The present invention relates to a method for separation of a targeted molecular species from a mixture of two or more molecular species by a sublimation system under a specific condition. FIG. 1 shows a sectional view of a sublimation system 1. The sublimation system 1 comprises (A) a thermal sublimation unit 10 with a linear tube 11 and (B) a thermal control units 20 which control temperatures of the thermal sublimation unit. The sublimation system optionally comprises a vacuum pump 30 to decompress the inside of the thermal sublimation unit and a gas supply unit 31.

(A) Thermal Sublimation Unit

The thermal sublimation unit comprises a linear tube 11. The tube is sealed and can be evacuated using a vacuum pump 30. The tube can be made from any materials if the material has a sufficient thermal resistivity at the temperature of sublimation of organic molecules and does not react with organic molecules. Examples of the material include crystal, ceramics and metals. The thermal sublimation unit is surrounded by a multi-zone furnace 22 which heats multiple positions of the thermal sublimation unit controlled by the thermal control unit. The tube comprises two parts, (A-1) a sublimation part 12 and (A-2) a deposition part 13. The sublimation part is the area where the mixture is heated and sublimated. The mixture 81 can be put on a boat 80 located on the sublimation part. The sublimation part 12 is heated by a thermal control unit 20. The temperature of the sublimation part is at least higher than the temperature of sublimation temperature of the targeted molecular species. Preferably, the temperature is at least (10° C. higher than the sublimation temperature of the targeted molecular species. The temperature is less than decomposition temperature of the targeted molecular species. The molecular species included in the mixture are sublimated at the sublimation part and are diffused to the deposition part 13.

The deposition part 13 is the area where sublimated molecular species are deposited. The temperature of the deposition part is controlled to moderately decrease as the distance from the beginning of the deposition part. Molecular species are deposited in order of deposition temperatures for the molecular species. It means, the molecular species with the highest deposition temperature deposits first, then the molecular species with the second highest deposition temperature deposits next. In a deposition part 13, a molecular species initially deposits at their deposition temperatures, but it does not all deposit at the exact location of that temperature. The deposition of a molecular species in the deposition part has a width. In this application, it is called the deposition area of the molecular species (51a and 51b).

The position where the targeted molecular species initially deposits is position X (60). The temperature of the position X is deposition temperature of the targeted molecular species at a pressure in the tube 11. Deposition temperature at a specific pressure can be calculated from vapor pressure curve of the targeted molecular species. Same as above, the deposition area of the targeted molecular species also has a width (50). The position where the deposition of the targeted molecular species is ended is described as position Y (61).

Inventors of this invention found that molecular species deposit down the tube following an exponential decay as function of distance. Inventors of this invention plotted the amount of deposited molecular species versus the distance from the beginning of the deposition part (deposition curve), and found an exponential function curve. The coefficient in the exponential function is the sticking coefficient, it is peculiar value for each molecular species that is independent of pressure, temperature and diameter of a sublimation tube. It is also independent from a material of a liner tube, and a roughness of a surface of a tube does not impact the sticking coefficient. Based on the above new findings, the inventors of this invention developed a method to effectively separate the targeted molecular species from a mixture, calculating the deposition areas of molecular species.

The exponential function is described as the following formula (1).

N₍ₓ₎=N₍₀₎e^{-αx} (1)

In the formula (1), x is a distance from beginning of deposition of a molecular species to be calculated, N(0) is the amount of material deposited at x=0, N(x) is the amount of material deposited at the distance x and α is the sticking coefficient of the molecular species. The unit of distance x can be centimeters (cm), and the unit of the amount of deposited material can be grams (g).

Using formula (1), deposition areas of each molecular species can be calculated. Example is that: a sticking coefficient of a molecular species is determined by plotting deposition curve for the molecular species. If setting N(x)/N(0) is 0.0001, 99.99% of the molecular species is deposited at the distance x. To insert those numbers in formula (1), the distance x can be calculated. This allows the length of the deposition area to be calculated for deposition of 99.99% of the molecular species.

To obtain the targeted molecular species with high purity, the deposition area of the targeted molecular species should not overlap with deposition areas of other molecular species. It can be achieved by adjusting the thermal gradient of the deposition part. Referring to FIG. 1, position X (60) should be a suitable distance away from the deposition area 51a which is the deposition area of a molecular species with higher deposition temperature. Preferably, the position 62 of the end of the deposition area and position X should be separated 5% or more based on the whole length of the tube. At the same time, position Y (61) should be at least far enough from the beginning of the deposition area 51b which is the deposition area of a molecular species with lower deposition temperature. It means the temperature at the position Y is higher than the deposition temperature of molecular species with lower deposition temperatures than the targeted molecular species. Preferably, position Y and the position 63 (the position of the beginning of the deposition area 51b) should be separated 5% or more based on the whole length of the tube. To set thermal gradient of the deposition area as the condition disclosed above, the obtained targeted molecular species has quite high purity, i.e. 99.99% or more.

(B) Thermal Control Units

The thermal control units 20 comprises of multi-zone furnace 22, thermocouples 21 and a means to heat the thermocouples. The thermocouples control the temperatures of each zone of the multi-zone furnace surrounding to the linear tube 11. The multi-zone furnace is located near the outside of the liner tube, or contacted to the outside of the liner tube. The thermocouple located at the multi-zone furnace of the sublimation part controls the temperature of the sublimation part, while the thermocouples located at the multi-zone furnaces of the deposition part control the temperature of the deposition part so that the temperature moderately decreases and the distance away from the sublimation part increases. By controlling the thermal gradient of the deposition part sufficiently, a separation of the targeted organic molecule with quite isolated high purity is achieved.

Another aspect of the invention is a method for separating two or more molecular species from a mixture of the molecular species by sublimation. The same sublimation system described above can be used for the invention. The method comprises seven steps.

The first step is, (a) calculating deposition temperatures for each of the molecular species at a particular pressure from vapor pressure curves of each molecular species, and determining the order between the molecular species in order of decreasing deposition temperature. As disclosed above, the relationship between pressures versus deposition temperatures is known as a vapor pressure curve for each molecular species. Therefore, the deposition temperature of a molecular species at a particular pressure can be calculated by the vapor pressure curve of the molecular species. After the vapor pressure curves for all molecular species included in a mixture are calculated or determined, the order of the higher deposition temperature is determined. The molecular species are deposited in a deposition unit 13, in order of decreasing deposition temperature.

The second step is, (b) calculating sticking coefficient of a molecular species which has the highest deposition temperature within a mixture to be separated. When the mixture comprises three molecular species, the sticking coefficient of the molecular species with the highest deposition temperature within the three is calculated. The sticking coefficient can be calculated as disclosed above.

The third step is, (c) calculating a deposition distance for the molecular species with the highest deposition temperature from formula (1).

N₍ₓ₎=N₍₀₎e^{-αx} (1)

wherein the formula (1), x is a distance from beginning of deposition for the molecular species, N(0) is the amount of material deposited at x=0, N(x) is the amount of material deposited at the distance x, and α is the sticking coefficient of the molecular species. The method to calculate the distance x is disclosed above. Preferably, N(x)/N(0) is equal to or greater than 0.0001 and this ratio is dependent on the purity necessary for the isolated target compound.

The fourth step is, (d) determine a deposition point for the molecular species which has the next highest deposition temperature, wherein the deposition point is the necessary distance from the deposition area of the previously depositing material initial deposition temperature. As disclosed above, the deposition area of the highest deposition temperature has a width, and it is calculated in step (c). On the other hand, the deposition temperature of the molecular species with the second highest deposition temperature is calculated in the step (a). Therefore, the thermal gradient is controlled so that the deposition of the molecular species with the second highest deposition temperature is far enough from the end of the deposition area of the first deposited molecular species. Preferably, the position of the end of the first deposition area and the position of the beginning of the second deposition area (the deposition area of the molecular species with the second highest deposition temperature) should be separated 5% or more based on the whole length of the tube.

The fifth step is, (e) repeating steps (b) to (d) for the next molecular species which has the second highest deposition temperature. Those steps are repeated until deposition point for all molecular species are determined. Therefore, if a mixture contains three molecular species, deposition distance of a molecular species with the second highest deposition temperature is calculated from its sticking coefficient and deposition temperature, then the deposition point of the last molecular species is determined.

The sixth step is, (f) setting temperatures of each deposition point based on the deposition temperatures of the corresponding molecular species.

Then, (g) sublimation separation is conducted based on the determined thermal gradients.

### EXAMPLES

### Example 1

### Comparative Example

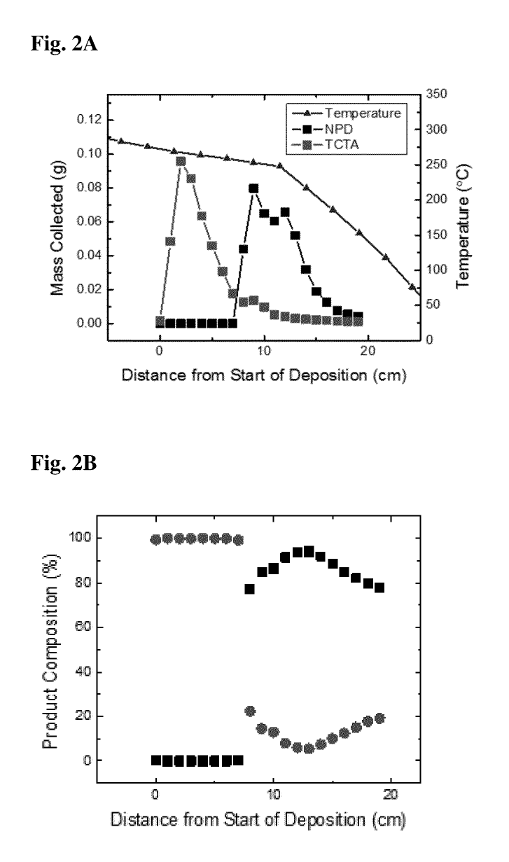

A linear thermal sublimation apparatus consisting of a quartz collection tube (diameter was 30 mm, the length of heated zone was 500 mm) in a variable temperature furnace was charged with a source boat containing an equal mass mixture of N,N′-Di(1-naphthyl)-N,N′-diphenyl-(1,1′-biphenyl)-4,4′-diamine (NPD) and 4,4′,4″-Tri-9-carbazolytriphenylamine (TCTA). The tube was evacuated to a base pressure of 10⁻⁷ torr and heated such that the area of the tube where the source boat was located was 310° C. The deposition zone temperature was operated such that the onset of mass deposition was 260° C. and decreased at a rate of 1.6° C./cm for a distance of 12 cm and then allowed to rapidly cool uncontrollable. The resulting deposition region is shown in FIG. 2A and FIG. 2B. Depicting significant co-deposition of both NPD and TCTA that resulted in low mass recovery (a) of both components in relatively low purity (b).

### Example 2

### Inventive Example

### (1) Determine Deposition Temperature of NPD and TCTA

From vapor pressure curve of TCTA and NPD, the deposition temperature of those chemicals at 10⁻⁷ torr were calculated to around 260° C. and around 240° C. respectively.

### (2) Determine a Sticking Coefficient of TCTA

The sticking coefficient of TCTA was determined by subliming a pure feed of TCTA in the same sublimation equipment described in Example 1. The tube was evacuated to 10⁻⁷ torr and heated such that the area of the tube where the source boat was located was 310° C. and the deposition zone was set a lower temperature to induce a convective flow of material. The sublimed TCTA was found to initially deposit at the point in the deposition area where the temperature was 260° C. The mass of TCTA deposited in the deposition area was monitored as a function of distance down the tube from the initial deposition location and this data was fit to formula (1) to solve for the sticking coefficient α. The sticking coefficient of the TCTA was determined as 0.32.

### (3) Calculating Deposition Distance of TCTA by Formula (1)

N₍ₓ₎=N₍₀₎e^{-αx} (1)

N(x)/N(0)=0.0001, α=0.32 were inserted in the formula (1).

### N(x)/N(0)=exp(−0.32*x)

x=ln [0.0001/(−0.32)]=about 29 (cm)
Therefore, the deposition distance of TCTA was calculated as 29 cm. To add 5% of margin, the distance between the point of the end of deposition area of TCTA and the point of the beginning of deposition area of NPD was determined as 1 cm.

### (4) Separation of NPD and TCTA

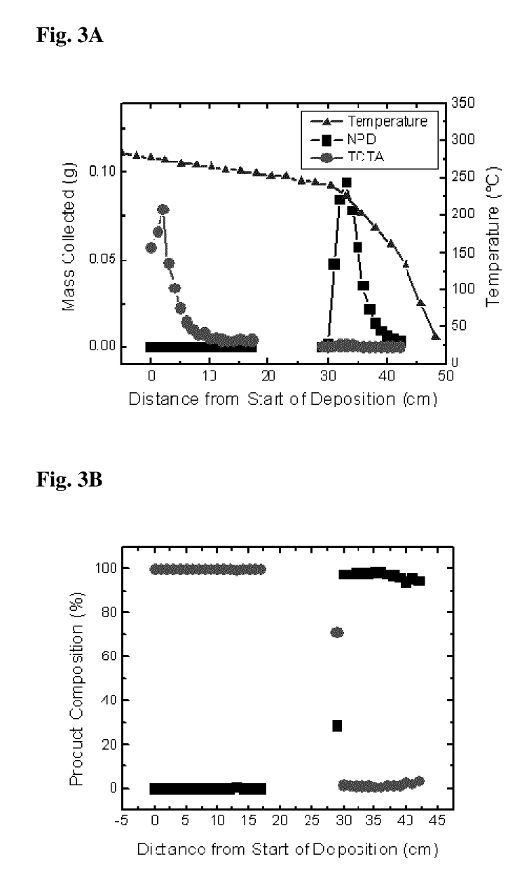

The same procedure as of Example 1 was conducted excepting for the thermal gradient was set as decreased at a rate of 0.7° C./cm for a distance of 30 cm. The resulting deposition region is shown in FIG. 3A and FIG. 3B. The deposition region shown in FIG. 3A and FIG. 3B depict effective separation of NPD and TCTA that resulted in good mass recovery (a) of both components in relatively high purity (b).

1. A method for separation of a targeted molecular species from a mixture of two or more molecular species by a sublimation system comprising
(A) a thermal sublimation unit with a linear tube, comprising (A-1) a sublimation part where the mixture is heated and sublimated and (A-2) a deposition part where sublimated molecular species are deposited, in which the targeted molecular species is deposited in a specific area between position X and position Y in the deposition part and
(B) thermal control units which control temperatures of multiple positions of the thermal sublimation unit to decrease as the distance from the sublimate part increases,
wherein position X, position Y and temperatures of the thermal sublimation unit are controlled to satisfy the following (i) to (iii):
(i) the temperature of the sublimation part is at least higher than the sublimation temperature of the targeted molecular species,
(ii) position X is away from deposition areas of molecular species with higher deposition temperatures than the targeted molecular species, and the temperature at the position X is set at the deposition temperature of the targeted molecular species,
(iii) the position Y is decided from deposition area of the targeted molecular species, and the temperature at the position Y is higher than deposition temperatures of molecular species with lower deposition temperatures than the targeted molecular species.

2. The method of claim 1, wherein the deposition areas are calculated by deposition distances for each of molecular species using formula (1)
N₍ₓ₎=N₍₀₎e^{-αx} (1)
wherein x is a distance from beginning of deposition for a molecular species to be calculated, N(0) is the amount of material deposited at x=0, N(x) is the amount of material deposited at the distance x and α is the sticking coefficient of the molecular species.

3. The method of claim 1 or 2, wherein the molecular species is a small organic molecular species.

4. The method according to any of claims 1 to 3, wherein the deposition distances are determined at the point of N(x)/N(0) equal to or smaller than 0.0001.

5. The method according to any of claims 1 to 4, wherein the purity of the targeted molecular species obtained by the method is 99.99% or higher.

6. A method for separation of two or more molecular species from a mixture of the molecular species by sublimation, wherein the method comprises the steps of:
(a) calculating deposition temperatures for each of the molecular species at a particular pressure from vapor pressure curves of each molecular species, and determining the order between the molecular species in the order of decreasing deposition temperatures,
(b) calculating sticking coefficient of a molecular species which has the highest deposition temperature within a mixture to be separated,
(c) calculating a deposition distance for the molecular species with the highest deposition temperature from formula (1)
N₍ₓ₎=N₍₀₎e^{-αx} (1)
wherein x is a distance from beginning of deposition for the organic molecule, N(0) is the amount of material deposited at x=0, N(x) is the amount of material deposited at the distance x, and α is the sticking coefficient of the molecular species,
(d) determine a deposition point for the molecular species which has the next highest deposition temperature, wherein the deposition point is away from at least the deposition distance of the molecular species with the highest deposition temperature,
(e) repeating steps (b) to (d) for the next molecular species which has the next highest deposition temperature until deposition points for all molecular species are determined,
(f) setting temperatures of each deposition point based on the deposition temperatures of the corresponding molecular species, then
(g) conducting separation by sublimation.

7. The method of claim 6, wherein the deposition distance is determined at the point of N(x)/N(0) equal to or smaller than 0.0001.
